(19) United States
(12) Patent Application Publication
(10) Pub. No.: US 2024/0389658 A1
(43) Pub. Date: Nov. 28, 2024

(54) AEROSOL DELIVERY COMPONENT

(71) Applicant: Imperial Tobacco Limited, Bristol (GB)

(72) Inventors: Peter Lomas, Liverpool Merseyside (GB); Molly McGuinness, Liverpool Merseyside (GB)

(21) Appl. No.: 18/691,446

(22) PCT Filed: Sep. 9, 2022
(86) PCT No.: PCT/EP2022/075172

§ 371 Date: Mar. 12, 2024

(30) Foreign Application Priority Data
Sep. 15, 2021 (EP) 21196985.2

(51) Int. Cl. A24F 40/46; A24F 40/70; H05B 3/54

(52) U.S. Cl. CPC A24F 40/46; A24F 40/70; H05B 3/54

(57) ABSTRACT

Disclosed is a vaporiser electrical contact pin ( )200 for an aerosol delivery component (104) comprising a main body (210) comprising a first longitudinal end face (211); and a transverse groove (230) in the first longitudinal end face (211) for fixing a heating wire (152) of the vaporiser (100). Also disclosed is an aerosol delivery component (104) comprising an electrical contact pin (200) having a transverse groove (230) and a vaporiser heating wire (152) wherein the vaporiser heating wire is fixed within the transverse groove.

### TECHNICAL FIELD

The present disclosure relates to an aerosol-delivery component (e.g. a smoking substitute component), which may be a consumable for receipt in an aerosol-delivery device to form an aerosol-delivery system (e.g. a smoking substitute system).

### BACKGROUND

The smoking of tobacco is generally considered to expose a smoker to potentially harmful substances. It is generally thought that a significant amount of the potentially harmful substances are generated through the heat caused by the burning and/or combustion of the tobacco and the constituents of the burnt tobacco in the tobacco smoke itself.

Combustion of organic material such as tobacco is known to produce tar and other potentially harmful by-products. There have been proposed various smoking substitute systems in order to avoid the smoking of tobacco.

Such smoking substitute systems can form part of nicotine replacement therapies aimed at people who wish to stop smoking and overcome a dependence on nicotine.

Smoking substitute systems, which may also be known as electronic nicotine delivery systems, may comprise electronic systems that permit a user to simulate the act of smoking by producing an aerosol, also referred to as a “vapour”, which is drawn into the lungs through the mouth (inhaled) and then exhaled. The inhaled aerosol typically bears nicotine and/or flavourings without, or with fewer of, the odour and health risks associated with traditional smoking.

In general, smoking substitute systems are intended to provide a substitute for the rituals of smoking, whilst providing the user with a similar experience and satisfaction to those experienced with traditional smoking and tobacco products.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories. Some smoking substitute systems are designed to resemble a traditional cigarette and are cylindrical in form with a mouthpiece at one end. Other smoking substitute systems do not generally resemble a cigarette (for example, the smoking substitute device may have a generally box-like form).

There are a number of different categories of smoking substitute systems, each utilising a different smoking substitute approach. A smoking substitute approach corresponds to the manner in which the substitute system operates for a user.

One approach for a smoking substitute system is the so-called “vaping” approach, in which a vaporisable liquid, typically referred to (and referred to herein) as “e-liquid”, is heated by a heater to produce an aerosol vapour which is inhaled by a user. An e-liquid typically includes a base liquid as well as nicotine and/or flavourings. The resulting vapour therefore typically contains nicotine and/or flavourings. The base liquid may include propylene glycol and/or vegetable glycerine.

A typical vaping smoking substitute system includes a mouthpiece, a power source (typically a battery), a tank or liquid reservoir for containing e-liquid, as well as a heater. In use, electrical energy is supplied from the power source to the heater, which heats the e-liquid to produce an aerosol (or “vapour”) which is inhaled by a user through the mouthpiece.

Vaping smoking substitute systems can be configured in a variety of ways. For example, there are “closed system” vaping smoking substitute systems which typically have a heater and a sealed tank which is pre-filled with e-liquid and is not intended to be refilled by an end user. One subset of closed system vaping smoking substitute systems include a device which includes the power source, wherein the device is configured to be physically and electrically coupled to a consumable component including the tank and the heater. In this way, when the tank of the consumable component has been emptied, the device can be reused by connecting it to a new consumable component. Another subset of closed system vaping smoking substitute systems are completely disposable, and intended for one-use only.

There are also “open system” vaping smoking substitute systems which typically have a tank that is configured to be refilled by a user, so the system can be used multiple times.

An example vaping smoking substitute system is the myblu™ e-cigarette. The myblu™ e cigarette is a closed system which includes a device and a consumable component. The device and consumable component are physically and electrically coupled together by pushing the consumable component into the device. The device includes a rechargeable battery. The consumable component includes a mouthpiece, a sealed tank which contains e-liquid, as well as a vaporiser, which for this system is a heating filament (heating wire) coiled around a portion of a wick which is partially immersed in the e-liquid. The system is activated when a microprocessor on board the device detects a user inhaling through the mouthpiece. When the system is activated, electrical energy is supplied from the power source to the vaporiser, which heats e-liquid from the tank to produce a vapour which is inhaled by a user through the mouthpiece.

Another example vaping smoking substitute system is the blu PRO™ e-cigarette. The blu PRO™ e cigarette is an open system which includes a device, a (refillable) tank, and a mouthpiece. The device and tank are physically and electrically coupled together by screwing one to the other. The mouthpiece and refillable tank are physically coupled together by screwing one into the other, and detaching the mouthpiece from the refillable tank allows the tank to be refilled with e-liquid. The system is activated by a button on the device. When the system is activated, electrical energy is supplied from the power source to a vaporiser, which heats e-liquid from the tank to produce a vapour which is inhaled by a user through the mouthpiece.

As the vapour passes through the consumable (entrained in the airflow) from the location of vaporization to an outlet of the consumable (e.g. a mouthpiece), the vapour cools and condenses to form an aerosol for inhalation by the user. The aerosol may contain nicotine and/or flavour compounds.

The vaporiser of many smoking substitute systems comprises a heating filament or heating wire which is electrically connected to a power source and is heated by electrical energy in order to vaporise the e-liquid. In smoking substitute systems comprising a component which has a vaporiser, the heating filament or heating wire can be connected to electrical contact elements in the component. These electrical contact elements provide the electrical connection between the component and a device containing a power source as described above.

For example, the myblu™ e-cigarette comprises flat contact plates which are electrically connected to each end of the heating filament. To allow the contact plates to be assembled within the consumable component, small slits are provided in the base of the component. These slits are a potential site of e-liquid leakage from the consumable component. Leakage of the e-liquid is undesirable as the user risks inhaling un-vaporised e-liquid which may result in an unpleasant sensation in the mouth of the user. In addition, e-liquid leakage may be wasteful or costly to the user.

Given the above, it is desirable to provide improved electrical contact elements within a component of a smoking substitute system which may be assembled in such a way as to reduce leakage of e-liquid from the component.

### SUMMARY

According to a first aspect there is provided a vaporiser electrical contact pin for an aerosol delivery component comprising: a main body comprising a first longitudinal end face; and a transverse groove in the first longitudinal end face for fixing a heating wire of the vaporiser.

By providing a vaporiser electrical contact pin (i.e. an elongate rod-like element as opposed to a contact plate), it may be assembled into the aerosol delivery component using an interference fit (i.e. a press fit). The contact pin can be press-fitted into a hole which matches the cross-sectional shape of the electrical contact pin which may reduce the likelihood of e-liquid leakage. Further, press fitting the electrical contact pin into the component may reduce the complexity of the assembly process.

Optional features will now be set out. These are applicable singly or in any combination with any aspect.

In some embodiments, the transverse groove may extend in a transverse direction substantially perpendicular to a longitudinal axis of the main body of the electrical contact pin. The transverse groove may have an elongate groove axis which extends the length of the transverse groove in the transverse direction. The elongate groove axis may be substantially perpendicular to the longitudinal axis of the main body.

The transverse groove may extend in the transverse direction from a first edge portion of the first longitudinal end face. In some embodiments, the first longitudinal end face may have a curved first edge portion i.e. the first edge portion may comprise an arc. For example, the first longitudinal end face may be a circle or oval. In these embodiments, the elongate groove axis may be perpendicular to a tangent to the arc on the first edge portion of the first longitudinal end face.

The transverse groove may extend fully across a transverse width of the first longitudinal end face. In other words the elongate groove axis may extend from the first edge portion of the first longitudinal end face to a second edge portion opposite (e.g. diametrically opposite) the first edge portion. For example, in embodiments where the first longitudinal end face is a circle, the transverse groove may extend fully across the diameter of the first longitudinal end face.

The transverse groove may have a depth which extends from the first longitudinal end face in a longitudinal direction parallel to the longitudinal axis of the main body. The depth of the transverse groove may be between about 0.5 mm and about 1.5 mm e.g. the depth of the transverse groove may be about 1 mm. The transverse groove may comprise a base which is spaced from the first longitudinal end face in the longitudinal direction. The transverse groove may comprise laterally spaced opposing first and second side walls extending from the first longitudinal end face to the base of the transverse groove.

The transverse groove may comprise first edge which is formed by the first longitudinal end face of the main body meeting the first side wall. The transverse groove may comprise second edge which is formed by the first longitudinal end face of the main body meeting the second side wall. The transverse groove may comprise third edge which is formed by the first side wall meeting the base of the transverse groove. The transverse groove may comprise a fourth edge which is formed by the second side wall meeting the base of the transverse groove. Therefore, the first and second edges are spaced laterally from each other. Likewise, the third and fourth edges are spaced laterally from each other. Any one of the first, second, third or fourth edges may be parallel with any other one of the first, second, third or fourth edges.

In some embodiments, the first and second side walls are moveable between a receiving position and a fixing position. In the fixing position the first and second edges are spaced closer together than in the receiving position. In other words, the first side wall may pivot/hinge about the third edge and the second side wall may pivot/hinge about the fourth edge. This means that the heating wire (heating filament) may be more easily placed in the transverse groove when the transverse groove is in the receiving position and then held securely in the transverse groove when in the fixing position. This may simplify the process of assembling the heating wire with the electrical contact pin and may improve the stability of the connection between the heating wire and the electrical contact pin.

In the receiving position, the transverse groove may be tapered such that the first and second side walls are spaced further apart at the longitudinal end face of the main body than at the base of the transverse groove. In other words, the side walls of the transverse groove may slope inwards towards the longitudinal axis of the main body. In the receiving position, the side walls of the transverse groove may slope inwards towards the longitudinal axis of the main body at a constant angle.

For example, the first and second edges of the transverse groove may be spaced laterally apart by between about 0.2 mm and about 0.4 mm and the third and fourth edges of the transverse groove may be spaced laterally by between about 0.1 mm and about 0.3 mm. More specifically, the first and second edges may be spaced laterally by between about 0.25 mm and about 0.35 mm and the third and fourth edges may be spaced by between 0.15 mm and about 0.25 mm e.g. the first and second edges may be spaced laterally by about 0.3 mm and the third and fourth edges may be spaced laterally by about 0.2 mm.

A tapered transverse groove may allow the ends of the heating wire to be fitted more easily into the electrical contact pins.

The first edge may comprise a first chamfered portion. The first chamfered portion may extend the full length of the first edge portion. The first chamfered portion may have an angle of between 30 degrees and 60 degrees from the first longitudinal end face e.g. the first chamfered portion may have an angle of 45 degrees from the first longitudinal end face. The first chamfered portion may have a chamfer distance of between 0.05 mm and 0.3 mm in the longitudinal direction e.g. the first chamfered portion may have a chamfer distance of 0.15 mm in the longitudinal direction. The second edge may comprise a second chamfered portion. The second chamfered portion may extend the full length of the second edge portion. The second chamfered portion may have an angle of between 30 degrees and 60 degrees from the first longitudinal end face e.g. the second chamfered portion may have an angle of 45 degrees from the first longitudinal end face. The second chamfered portion may have a chamfer distance of between 0.05 mm and 0.3 mm in the longitudinal direction e.g. the second chamfered portion may have a chamfer distance of 0.15 mm in the longitudinal direction. The first and second chamfered portions improve the fitting of the heating wire into the transverse groove.

The main body of the electrical contact pin may be a cylindrical body. It may have any transverse (i.e. transverse to the longitudinal axis) cross-sectional profile. It may have a substantially circular or oval transverse cross-section. As such, the longitudinal end face may be a circular or oval end face as described previously. It may be a solid body or it may be a hollow body e.g. with a hollow core which may extend axially.

The main body of the electrical contact pin may comprise a partition groove on one or more transverse faces of the main body. The partition groove may extend at least partly e.g. continuously around the outside of the main body. In embodiments where the cross-sectional shape of the main body is a circle or oval, the partition groove may be a circumferential partition groove. The partition groove may be spaced from the first longitudinal end face in the longitudinal direction. The partition groove may be spaced from the first longitudinal end face by between about 0.5 mm and 1.5 mm e.g. the partition groove may be spaced from the first longitudinal end face by about 1.0 mm.

The partition groove may have a depth which extends inwards from the or each transverse side (e.g. circumference) of the main body in a direction substantially perpendicular to the longitudinal axis of the main body. The depth of the partition groove may be between about 0.2 mm and about 0.3 mm e.g. the depth of the partition groove may be about 0.24 mm. The partition groove may comprise opposed upper and lower walls which are spaced apart in the longitudinal direction. The upper and lower walls may be spaced by between about 0.1 mm and about 0.5 mm e.g. the upper and lower walls may be spaced by about 0.2 mm. The upper and lower walls may be substantially parallel to the first longitudinal end face.

The base of the transverse groove may be aligned with the partition groove in the longitudinal direction. For example, the base of the transverse groove may be aligned with the upper or lower wall of the partition groove in the longitudinal direction (i.e. the base of the transverse groove may be substantially coplanar with either the upper or lower wall of the partition groove). The upper wall is more proximate the first longitudinal end face than the lower wall. In some embodiments, the base of the transverse groove may be aligned with the lower wall of the partition groove (i.e. the base of the transverse groove may be substantially coplanar with the lower wall of the partition groove).

The partition groove may create a weaker region at the base of the transverse groove. This may allow the transverse groove to be moved from the receiving position to the fixing position more easily in order to fix the heating wire in the transverse groove.

In some embodiments, the electrical contact pin may comprise a rotational location feature. This is so that the electrical contact pin can only be inserted into the component of the smoking substitute system in one orientation. As a result, the transverse groove is only orientated in one direction which may ensure that the transverse groove aligns correctly with the heating wire. This may simplify the assembly of the contact pins with the component.

In embodiments where the cross-sectional shape of the main body is a circle or oval, the rotational location feature may comprise a flattened surface on a transverse side of the main body. In other words, the rotational location feature may be a portion of the main body wherein the cross-sectional shape resembles a circle/oval which has had a circular/oval segment removed i.e. the cross-sectional shape of the rotational location feature portion may be approximately resemble a D-shape.

The main body may have a uniform transverse cross-section portion (e.g. a lower uniform transverse cross-section portion) having a constant transverse cross-sectional area and a frustum portion (e.g. an upper frustum portion) having transverse cross-sectional area which increases in the longitudinal direction (away from the first longitudinal end face). The uniform portion and the frustum portion are axially aligned i.e. a longitudinal axis of the uniform portion is substantially collinear with a longitudinal axis of the frustum portion.

The frustum portion may have a first frustum (longitudinal) end face. The first frustum end face may be the first longitudinal end face of the main body of the electrical contact pin. The frustum portion may have a largest transverse cross-sectional area at a second frustum (longitudinal) end longitudinally opposite the first frustum (longitudinal) end face. The transverse cross-sectional area at the second frustum end may have the same area as the transverse cross-sectional area of the uniform portion. The transverse cross-sectional shape at the second frustum end may have the same shape as the transverse cross-sectional shape of the uniform portion.

The uniform portion has a length in the longitudinal direction. The length of the uniform portion may be between about 2.5 mm and 4.5 mm e.g. the length of the uniform portion may be about 3.64 mm. The frustum portion has a length in the longitudinal direction. The length of the frustum portion may be between about 1.5 mm to about 3.5 mm e.g. the length of the frustum portion may be about 2.37 mm. The frustum portion has a slant angle between its longitudinal axis and a side surface. The slant angle may be between about 7.5 degrees and 15 degrees e.g. the slant angle may be about 11 degrees.

The frustum portion may be a truncated cone. The uniform portion may be a cylinder. The frustum portion may comprise the partition groove. In embodiments where the frustum portion is a truncated cone, the truncated cone may comprise the partition groove.

The uniform portion may have a first diameter e.g. in embodiments where the uniform portion is a cylinder. The first diameter may be between about 1.5 mm and about 2.5 mm. More specifically, the first diameter may be between about 2.05 mm and about 2.1 mm e.g. the first diameter may be about 2.05 mm.

In some embodiments, the first longitudinal end face may be a circle e.g. in embodiments where the frustum portion is a truncated cone. The first longitudinal end face may have a second diameter. The second diameter may be smaller than the first diameter. The second diameter may be between about 0.5 mm and about 1.5 mm e.g. the second diameter may be about 1.1 mm.

The main body of the electrical contact pin may comprise second longitudinal end face at an opposite end of the main body to the first longitudinal end face. In some embodiments, the main body of the electrical contact pin comprises a radially protruding flange proximate the second longitudinal end face. More specifically, the uniform portion may comprise a radially protruding flange proximate the second longitudinal end face.

In some embodiments, the flange may have a flange diameter (e.g. in embodiments where the uniform portion is a cylinder). The flange diameter may be larger than the first diameter. The flange diameter may be between about 2 mm and about 3 mm. More specifically, the flange diameter may be between about 2.48 mm and about 2.52 mm e.g. the flange diameter may be about 2.5 mm. The flange may have a length in the longitudinal direction. The length may be between about 0.1 mm and about 1 mm e.g. the length may be about 0.5 mm.

In some embodiments, the flange may comprise the rotational location feature. In some embodiments, the flange may comprise a flattened surface on a side of the flange. In other words, the rotational location feature may be the flange which has a cross-sectional shape resembling a circle with a circular segment removed i.e. the cross-sectional shape of the flange approximately resembles a D-shape. The flattened surface of the flange may be tangent to the uniform portion of the main body (e.g. in embodiments where the uniform portion is a cylinder. The flattened surface may extend in a direction parallel to the elongate groove axis. The flattened surface may extend in the longitudinal direction.

According to a second aspect there is provided an aerosol delivery component comprising an electrical contact pin according to the first aspect and a vaporiser heating wire, wherein the vaporiser heating wire is fixed within the transverse groove of the contact pin.

In some embodiments, the aerosol delivery component may comprise a plurality of electrical contact pins according to the first aspect. For example, the aerosol delivery component may comprise first and second electrical contact pins.

The vaporising heating wire may comprise a first end. The first end may be fixed within the transverse groove of the first electrical contact pin. The vaporising heating wire may comprise a second end. The second end may be fixed within the transverse groove of the second electrical contact pin.

The component may comprise a component housing having an upstream mouthpiece portion and a downstream base portion. The mouthpiece portion and base portion may be integrally formed.

The component comprises an airflow path that extends from an air inlet to an outlet. The outlet is preferably provided in the mouthpiece portion. In this respect, a user may draw fluid (e.g. air) into and along the airflow path by inhaling at the outlet (i.e. using the mouthpiece portion).

The air flow path passes the vaporiser between the air inlet and the outlet. The vaporiser may be housed in a vaporising chamber.

The airflow path may comprise a first portion extending from the air inlet towards the vaporiser. A second portion of the airflow path passes through the vaporising chamber to a conduit that extends to the outlet. The conduit may extend along the axial centre of the component.

References to “downstream” in relation to the airflow path are intended to refer to the direction towards the outlet/mouthpiece portion. Thus the second portion of the airflow path is downstream of the first portion of the airflow path. Conversely, references to “upstream” are intended to refer to the direction towards the air inlet. Thus the first portion of the airflow path (and the air inlet) is upstream of the second portion of the airflow path (and the outlet/mouthpiece portion).

References to “upper”, “lower”, “above” or “below” are intended to refer to the contact pin or the component when in an upright/vertical orientation i.e. with elongate (longitudinal/length) axis of the contact pin or component vertically aligned and with the mouthpiece vertically uppermost.

The component may comprise a tank for housing the aerosol precursor (e.g. a liquid aerosol precursor). The aerosol precursor may comprise an e-liquid, for example, comprising a base liquid and e.g. nicotine. The base liquid may include propylene glycol and/or vegetable glycerine.

The conduit may extend through the tank with the conduit walls defining an inner region of the tank. In this respect, the tank may surround the conduit e.g. the tank may be annular.

The tank may be defined by one or more side walls (e.g. laterally opposed first and second tank side walls) extending longitudinally from the mouthpiece portion.

The tank may further comprise opposing front and rear walls spaced by the laterally opposed first and second tank side walls.

At least a portion of one of the walls defining the tank may be translucent or transparent.

The distance between the first and second tank side walls may define a width of the tank. The distance between the front and rear walls may define a depth of the tank. The width of the tank may be greater than the depth of the tank.

The length of the tank/component housing may be greater than the width of the tank/component housing. The depth of the tank/component housing may be smaller than each of the width and the length.

The tank walls may be integrally formed and may additionally be integrally formed with the mouthpiece portion. In that way, the component may be easily manufactured using injection moulding.

As discussed above, the air flow path passes the vaporiser between the air inlet and the outlet. The vaporiser may comprise a heating element for heating a wick.

The wick may be oriented so as to extend in the direction of the width dimension of the component (perpendicular to the longitudinal axis of the component). Thus the wick may extend in a direction perpendicular to the direction of airflow in the airflow path.

The vaporiser may be disposed in the vaporising chamber. The vaporising chamber may form part of the airflow path.

The vaporising chamber may be defined by one or more chamber walls. The wick may be elongate and may extend between first and second opposing chamber walls. The first and second chamber walls may separate (i.e. partially separate) the vaporising chamber from the tank. The first and second chamber walls may each comprise a respective opening through which a respective end of the wick projects such that the wick is fluid communication with aerosol precursor/e-liquid in the tank. In this way a central portion of the wick may be exposed to air in the airflow path and end portions of the wick may be in contact with aerosol precursor/e-liquid stored in the tank. The wick may comprise a porous material. Thus, aerosol precursor may be drawn (e.g. by capillary action) along the wick, from the tank to the exposed portion of the wick.

A transverse chamber wall may separate the vaporising chamber from aerosol precursor in the tank. In this respect, the transverse chamber wall may partly define the (base of the) tank. A vent may be provided in the transverse chamber wall for the flow of air into the aerosol precursor tank (i.e. so as to allow for pressure equalisation in the tank).

The vaporising chamber may be defined by an insert (e.g. an insert at least partially formed of silicone) received into an open (e.g. lower) end of the housing. The chamber walls may be walls of the insert.

The wick may have an elongate shape. The wick may be cylindrical. The heating element may be in the form of a filament (wire) wound about the wick (e.g. the filament may extend helically about the wick). The filament may be wound about the exposed portion of the wick (i.e. the portion of the wick extending across the airflow path). The heating element may be electrically connectable (or connected) to a power source. Thus, in operation, the power source may supply electricity to (i.e. apply a voltage across) the heating element so as to heat the heating element. This may cause liquid stored in the wick (i.e. drawn from the tank) to be heated so as to form a vapour and become entrained in fluid flowing along the airflow path. This vapour may subsequently cool to form an aerosol in the airflow path (e.g. the third portion of the airflow path).

According to a third aspect there is provided a method of assembling an aerosol delivery component comprising an electrical contact pin according to the first aspect and a vaporiser heating wire, the method comprising: pushing a first end of the vaporiser heating wire into the transverse groove of the electrical contact pin; reducing a width of the transverse groove in order to fix the vaporiser heating wire in the electrical contact pin.

The method may further comprise press fitting the electrical contact pin into an aperture of the aerosol delivery component. This may be done before or after fixing the heating wire in the contact pin.

The step of reducing the width of the transverse groove may comprise crimping the contact pin e.g. crimping the portion of the contact pin above the partition groove.

The aerosol delivery component may be an aerosol delivery component according to the second aspect.

The electrical contact pin may further comprise a rotational location feature such that the method according to the third aspect may further comprise the initial step of rotationally locating the electrical contact pin relative to the aerosol delivery component according to the rotational location feature.

In a fourth aspect there is provided an aerosol-delivery system (e.g. a smoking substitute system) comprising a component according to the second aspect and an aerosol-delivery (e.g. smoking substitute) device.

The component may be an aerosol-delivery (e.g. a smoking substitute) consumable i.e. in some embodiments the component may be a consumable component for engagement with the aerosol-delivery (e.g. a smoking substitute) device to form the aerosol-delivery (e.g. a smoking substitute) system.

The device may be configured to receive the consumable component. For example the device and the consumable component may be configured to be physically coupled together. For example, the consumable component may be at least partially received in a recess of the device, such that there is snap engagement between the device and the consumable component. Alternatively, the device and the consumable component may be physically coupled together by screwing one onto the other, or through a bayonet fitting.

Thus, the consumable component may comprise one or more engagement portions for engaging with the device.

The consumable component may comprise an electrical interface for interfacing with a corresponding electrical interface of the device. One or both of the electrical interfaces may include one or more electrical contact pins according to the first aspect. Thus, when the device is engaged with the consumable component, the electrical interface may be configured to transfer electrical power from the power source to a heating element of the consumable component. The electrical interface may also be used to identify the consumable component from a list of known types. The electrical interface may additionally or alternatively be used to identify when the consumable component is connected to the device.

The device may alternatively or additionally be able to detect information about the consumable component via an RFID reader, a barcode or QR code reader. This interface may be able to identify a characteristic (e.g. a type) of the consumable. In this respect, the consumable component may include any one or more of an RFID chip, a barcode or QR code, or memory within which is an identifier and which can be interrogated via the interface.

In other embodiments, the component may be integrally formed with the aerosol-delivery (e.g. a smoking substitute) device to form the aerosol-delivery (e.g. s smoking substitute) system.

In such embodiments, the aerosol former (e.g. e-liquid) may be replenished by re-filling a tank that is integral with the device (rather than replacing the consumable). Access to the tank (for re-filling of the e-liquid) may be provided via e.g. an opening to the tank that is sealable with a closure (e.g. a cap).

Further features of the device are described below. These are applicable to both the device for receiving a consumable component and to the device integral with the component.

The device may comprise a power source e.g. a rechargeable battery. The device may comprise a controller.

A memory may be provided and may be operatively connected to the controller. The memory may include non-volatile memory. The memory may include instructions which, when implemented, cause the controller to perform certain tasks or steps of a method. The device may comprise a wireless interface, which may be configured to communicate wirelessly with another device, for example a mobile device, e.g. via Bluetooth®. To this end, the wireless interface could include a Bluetooth® antenna. Other wireless communication interfaces, e.g. WiFi®, are also possible. The wireless interface may also be configured to communicate wirelessly with a remote server.

An airflow (i.e. puff) sensor may be provided that is configured to detect a puff (i.e. inhalation from a user). The airflow sensor may be operatively connected to the controller so as to be able to provide a signal to the controller that is indicative of a puff state (i.e. puffing or not puffing). The airflow sensor may, for example, be in the form of a pressure sensor or an acoustic sensor. The controller may control power supply to a heating element in response to airflow detection by the sensor. The control may be in the form of activation of the heating element in response to a detected airflow. The airflow sensor may form part of the device.

In a fifth aspect there is provided a method of using the aerosol-delivery (e.g. smoking substitute) consumable component according to the second aspect, the method comprising engaging the consumable component with an aerosol-delivery (e.g. smoking substitute) device (as described above) having a power source so as to electrically connect the power source to the consumable component (i.e. to the vaporiser of the consumable component).

The invention includes the combination of the aspects and preferred features described except where such a combination is clearly impermissible or expressly avoided.

### BRIEF DESCRIPTION OF THE DRAWINGS

So that further aspects and features thereof may be appreciated, embodiments will now be discussed in further detail with reference to the accompanying figures, in which:

FIG. 1A is a front schematic view of a smoking substitute system;

FIG. 1B is a front schematic view of a device of the system;

FIG. 1C is a front schematic view of a consumable of the system;

FIG. 2A is a schematic of the components of the device;

FIG. 2B is a schematic of the components of the consumable;

FIG. 3 is a front section view of the consumable and showing the location of the vaporiser electrical contact pins within the consumable;

FIG. 4 is a perspective view of the vaporiser electrical contact pin.

FIG. 5 is a front detail view of the frustum portion of the vaporiser electrical contact pin shown in FIG. 4; and.

FIG. 6 is a top schematic view of the components of the consumable showing the heating wire assembled with two vaporiser electrical contact pins.

### DETAILED DESCRIPTION OF THE EMBODIMENTS

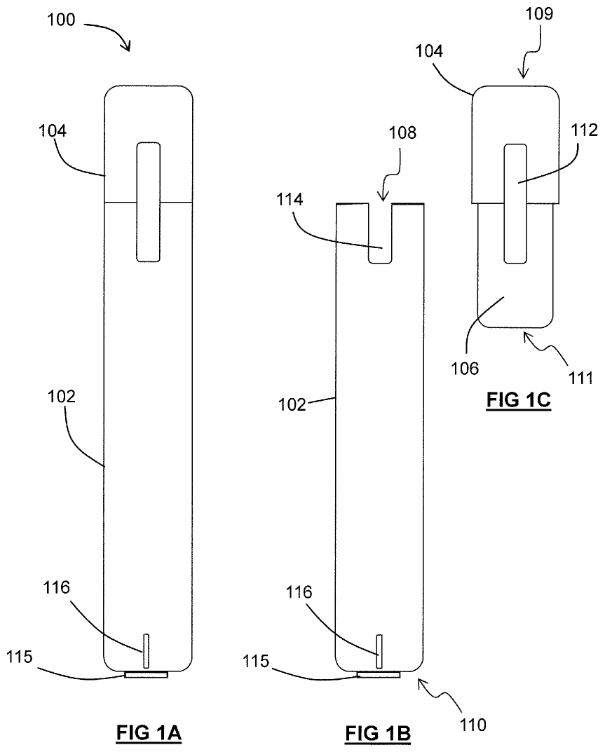

FIG. 1A shows a first embodiment of a smoking substitute system 100. In this example, the smoking substitute system 100 includes a device 102 and a component 104. The component 104 may alternatively be referred to as a “pod”, “cartridge” or “cartomizer”. It should be appreciated that in other examples (i.e. open systems), the device may be integral with the component. In such systems, a tank of the aerosol delivery system may be accessible for refilling the device.

In this example, the smoking substitute system 100 is a closed system vaping system, wherein the component 104 includes a sealed tank 106 and is intended for single-use only. The component 104 is removably engageable with the device 102 (i.e. for removal and replacement). FIG. 1A shows the smoking substitute system 100 with the device 102 physically coupled to the component 104, FIG. 1B shows the device 102 of the smoking substitute system 100 without the component 104, and FIG. 1C shows the component 104 of the smoking substitute system 100 without the device 102.

The device 102 and the component 104 are configured to be physically coupled together by pushing the component 104 into a cavity at an upper end 108 of the device 102, such that there is an interference fit between the device 102 and the component 104. In other examples, the device 102 and the component may be coupled by screwing one onto the other, or through a bayonet fitting.

The component 104 includes a mouthpiece portion at an upper end 109 of the component 104, and one or more air inlets (not shown) in fluid communication with the mouthpiece portion such that air can be drawn into and through the component 104 when a user inhales through the mouthpiece portion. The tank 106 containing e-liquid is located at the lower end 111 of the component 104.

The tank 106 includes a window 112, which allows the amount of e-liquid in the tank 106 to be visually assessed. The device 102 includes a slot 114 so that the window 112 of the component 104 can be seen whilst the rest of the tank 106 is obscured from view when the component 104 is inserted into the cavity at the upper end 108 of the device 102.

The lower end 110 of the device 102 also includes a light 116 (e.g. an LED) located behind a small translucent cover. The light 116 may be configured to illuminate when the smoking substitute system 100 is activated. Whilst not shown, the component 104 may identify itself to the device 102, via an electrical interface, RFID chip, or barcode.

The lower end 110 of the device 102 also includes a charging connection 115, which is usable to charge a battery within the device 102. The charging connection 115 can also be used to transfer data to and from the device, for example to update firmware thereon.

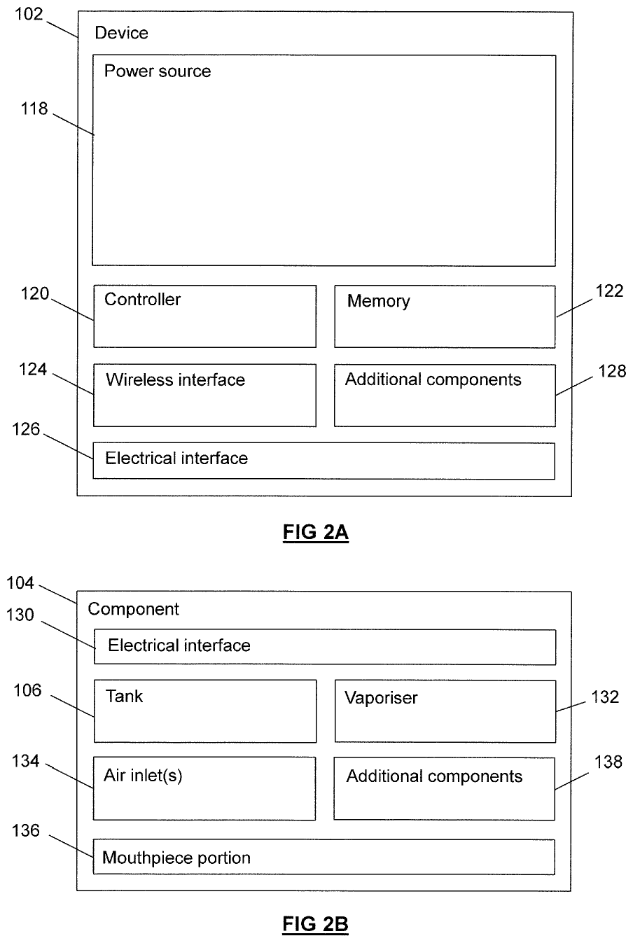

FIGS. 2A and 2B are schematic drawings of the device 102 and component 104. As is apparent from FIG. 2A, the device 102 includes a power source 118, a controller 120, a memory 122, a wireless interface 124, an electrical interface 126, and, optionally, one or more additional components 128.

The power source 118 is preferably a battery, more preferably a rechargeable battery. The controller 120 may include a microprocessor, for example. The memory 122 preferably includes non-volatile memory. The memory may include instructions which, when implemented, cause the controller 120 to perform certain tasks or steps of a method.

The wireless interface 124 is preferably configured to communicate wirelessly with another device, for example a mobile device, e.g. via Bluetooth®. To this end, the wireless interface 124 could include a Bluetooth® antenna. Other wireless communication interfaces, e.g. WiFi®, are also possible. The wireless interface 124 may also be configured to communicate wirelessly with a remote server.

The electrical interface 126 of the device 102 may include one or more electrical contacts. The electrical interface 126 may be located in a base of the aperture in the upper end 108 of the device 102. When the device 102 is physically coupled to the component 104, the electrical interface 126 is configured to transfer electrical power from the power source 118 to the component 104 (i.e. upon activation of the smoking substitute system 100).

The electrical interface 126 may also be used to identify the component 104 from a list of known components. For example, the component 104 may be a particular flavour and/or have a certain concentration of nicotine (which may be identified by the electrical interface 126). This can be indicated to the controller 120 of the device 102 when the component 104 is connected to the device 102. Additionally, or alternatively, there may be a separate communication interface provided in the device 102 and a corresponding communication interface in the component 104 such that, when connected, the component 104 can identify itself to the device 102.

The additional components 128 of the device 102 may comprise the light 116 discussed above.

The additional components 128 of the device 102 also comprises the charging connection 115 configured to receive power from the charging station (i.e. when the power source 118 is a rechargeable battery). This may be located at the lower end 110 of the device 102.

The additional components 128 of the device 102 may, if the power source 118 is a rechargeable battery, include a battery charging control circuit, for controlling the charging of the rechargeable battery. However, a battery charging control circuit could equally be located in a charging station (if present).

The additional components 128 of the device 102 may include a sensor, such as an airflow (i.e. puff) sensor for detecting airflow in the smoking substitute system 100, e.g. caused by a user inhaling through a mouthpiece portion 136 of the component 104. The smoking substitute system 100 may be configured to be activated when airflow is detected by the airflow sensor. This sensor could alternatively be included in the component 104. The airflow sensor can be used to determine, for example, how heavily a user draws on the mouthpiece or how many times a user draws on the mouthpiece in a particular time period.

The additional components 128 of the device 102 may include a user input, e.g. a button. The smoking substitute system 100 may be configured to be activated when a user interacts with the user input (e.g. presses the button). This provides an alternative to the airflow sensor as a mechanism for activating the smoking substitute system 100.

As shown in FIG. 2B, the component 104 includes the tank 106, an electrical interface 130, a vaporiser 132, one or more air inlets 134, a mouthpiece portion 136, and one or more additional components 138.

The electrical interface 130 of the component 104 may include one or more electrical contacts. The electrical interface 126 of the device 102 and an electrical interface 130 of the component 104 are configured to contact each other and thereby electrically couple the device 102 to the component 104 when the lower end 111 of the component 104 is inserted into the upper end 108 of the device 102 (as shown in FIG. 1A). In this way, electrical energy (e.g. in the form of an electrical current) is able to be supplied from the power source 118 in the device 102 to the vaporiser 132 in the component 104.

The vaporiser 132 is configured to heat and vaporise e-liquid contained in the tank 106 using electrical energy supplied from the power source 118. As will be described further below, the vaporiser 132 includes a heating filament and a wick. The wick draws e-liquid from the tank 106 and the heating filament heats the e-liquid to vaporise the e-liquid.

The one or more air inlets 134 are preferably configured to allow air to be drawn into the smoking substitute system 100, when a user inhales through the mouthpiece portion 136. When the component 104 is physically coupled to the device 102, the air inlets 134 receive air, which flows to the air inlets 134 along a gap between the device 102 and the lower end 111 of the component 104.

In operation, a user activates the smoking substitute system 100, e.g. through interaction with a user input forming part of the device 102 or by inhaling through the mouthpiece portion 136 as described above. Upon activation, the controller 120 may supply electrical energy from the power source 118 to the vaporiser 132 (via electrical interfaces 126, 130), which may cause the vaporiser 132 to heat e-liquid drawn from the tank 106 to produce a vapour which is inhaled by a user through the mouthpiece portion 136.

An example of one of the one or more additional components 138 of the component 104 is an interface for obtaining an identifier of the component 104. As discussed above, this interface may be, for example, an RFID reader, a barcode, a QR code reader, or an electronic interface which is able to identify the component. The component 104 may, therefore include any one or more of an RFID chip, a barcode or QR code, or memory within which is an identifier and which can be interrogated via the electronic interface in the device 102.

It should be appreciated that the smoking substitute system 100 shown in FIGS. 1A to 2B is just one exemplary implementation of a smoking substitute system. For example, the system could otherwise be in the form of an entirely disposable (single-use) system or an open system in which the tank is refillable (rather than replaceable).

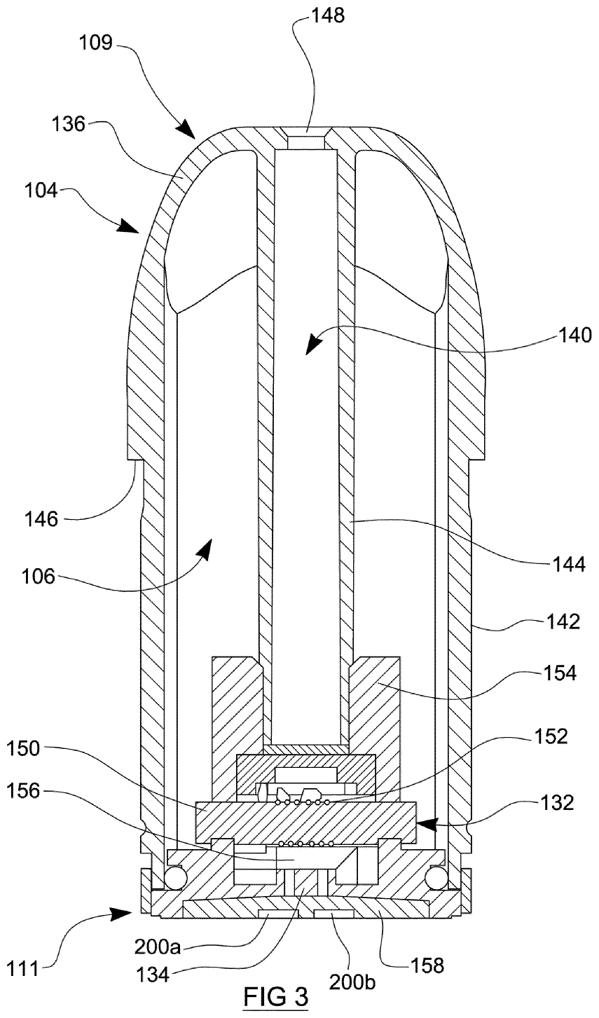

FIG. 3 is a section view of an example of the component 104 described above. The component 104 comprises a tank 106 for storing e-liquid, a mouthpiece portion 136 and a conduit 140 extending along a longitudinal axis of the component 104. In the illustrated embodiment the conduit 140 is in the form of a tube having a substantially circular transverse cross-section (i.e. transverse to the longitudinal axis). The tank 106 surrounds the conduit 140, such that the conduit 140 extends centrally through the tank 106.

A tank housing 142 of the tank 106 defines an outer casing of the component 104, whilst a conduit wall 144 defines the conduit 140. The tank housing 142 extends from the lower end 111 of the component 104 to the mouthpiece portion 136 at the upper end 109 of the component 104. At the junction between the mouthpiece portion 136 and the tank housing 142, the mouthpiece portion 136 is wider than the tank housing 142, so as to define a lip 146 that overhangs the tank housing 142. This lip 146 acts as a stop feature when the component 104 is inserted into the device 102 (i.e. by contact with an upper edge of the device 102).

The tank 106, the conduit 140 and the mouthpiece portion 136 are integrally formed with each other so as to form a single unitary component and may e.g. be formed by way of an injection moulding process. Such a component may be formed of a thermoplastic material such as polypropylene.

The mouthpiece portion 136 comprises a mouthpiece aperture 148 defining an outlet of the conduit 140. The vaporiser 132 is fluidly connected to the mouthpiece aperture 148 and is located in a vaporising chamber 156 of the component 104. The vaporising chamber 156 is downstream of the inlet 134 of the component 104 and is fluidly connected to the mouthpiece aperture 148 (i.e. outlet) by the conduit 140.

The vaporiser 132 comprises a porous wick 150 and a heating wire 152 coiled around the porous wick 150. The wick 150 extends transversely across the chamber vaporising 156 between sidewalls of the chamber 156 which form part of an inner sleeve 154 of an insert 158 that defines the lower end 111 of the component 104 that connects with the device 102. The insert 158 is inserted into an open lower end of the tank 106 so as to seal against the tank housing 142.

In this way, the inner sleeve 154 projects into the tank 106 and seals with the conduit 140 (around the conduit wall 144) so as to separate the vaporising chamber 156 from the e-liquid in the tank 106. Ends of the wick 150 project through apertures in the inner sleeve 154 and into the tank 106 so as to be in contact with the e-liquid in the tank 106. In this way, e-liquid is transported along the wick 150 (e.g. by capillary action) to a central portion of the wick 150 that is exposed to airflow through the vaporising chamber 156. The transported e-liquid is heated by the heating wire 152 (when activated e.g. by detection of inhalation), which causes the e-liquid to be vaporised and to be entrained in air flowing past the wick 150. This vaporised liquid may cool to form an aerosol in the conduit 140, which may then be inhaled by a user.

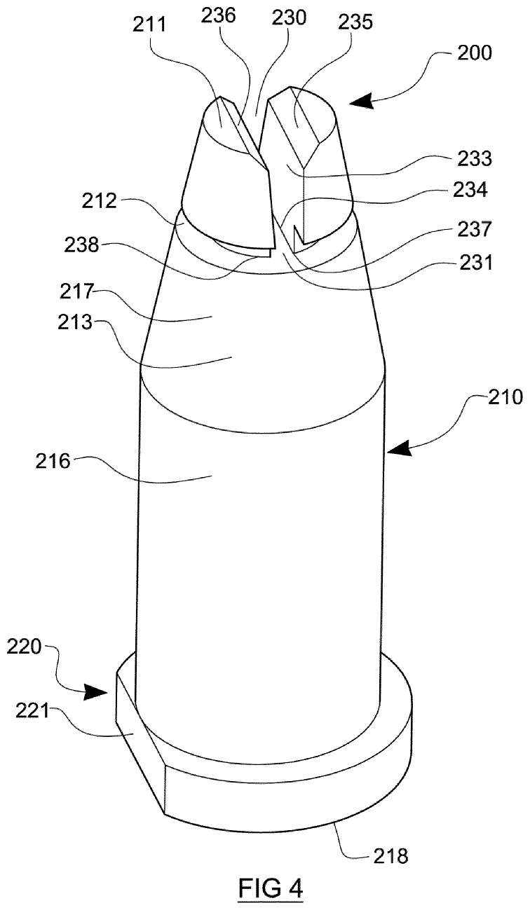

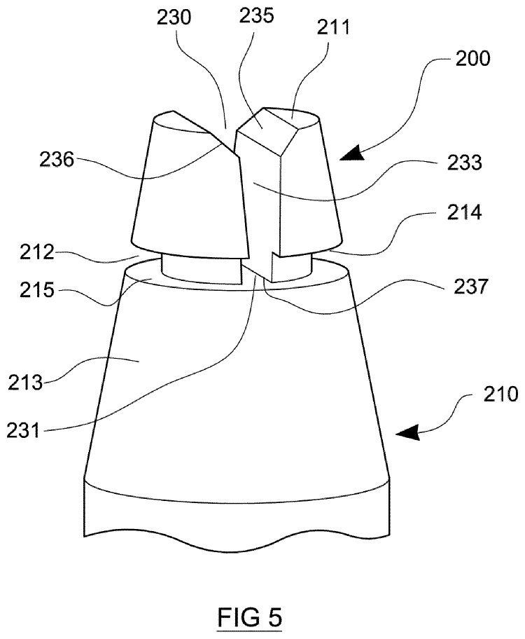

The insert also 158 accommodates the electrical interface 119 of the consumable component 102 comprising two electrical contacts 200a, 200b which are identical to the vaporiser electrical contact pin 200 shown in FIGS. 4 and 5. The two electrical contacts 200a, 200b are electrically connected to the heating wire 152. In this way, when the consumable component 104 is engaged with the device 102, power can be supplied from the power source 118 of the device to the heating wire 152.

FIGS. 4 and 5 show an exemplary embodiment of a vaporiser electrical contact pin 200. The electrical contact pin 200 comprises a main body 210 comprising a first longitudinal end face 211 and a transverse groove 230 in the first longitudinal end face 211 for fixing a heating wire 152 of the vaporiser 132.

The transverse groove 230 extends in a transverse direction substantially perpendicular to a longitudinal axis (a vertical axis in FIG. 5) of the main body 210 of the electrical contact pin 200. The transverse groove 230 has an elongate groove axis which extends the length of the transverse groove 230 in the transverse direction. The elongate groove axis is substantially perpendicular to the longitudinal axis of the main body 210.

The first longitudinal end face 211 is circular and the transverse groove 230 extends edge to edge across the diameter of the first longitudinal end face 211 through the centre of the circular first longitudinal end face 211. The transverse groove 230 has a base 231 which is spaced from the first longitudinal end face 211 in a longitudinal direction parallel to the longitudinal axis of the main body 210. Therefore, the transverse groove 230 has a depth of about 1 mm from the first longitudinal end face 211.

The transverse groove 230 also comprises a first side wall 233 and a second side wall 234 which are laterally spaced apart and extend from the first longitudinal end face 211 to the base 231 of the transverse groove 230.

The transverse groove 230 also comprises a first edge formed by the first longitudinal end face 211 meeting the first side wall 233 which comprises a first chamfered portion 235 extending the full length of the first edge. The transverse groove 230 also comprises second edge formed by the first longitudinal end face 211 meeting the second side wall 234 which comprises a second chamfered portion 236 extending the full length of the second edge. The first and second chamfered portions 235, 236 have a chamfer angle of 45 degrees and a chamfer distance of 0.15 mm.

The transverse groove comprises a third edge 237 which is formed by the first side wall 233 meeting the base 231 of the transverse groove 230. The transverse groove also comprises a fourth edge 238 which is formed by the second side wall 234 meeting the base 231 of the transverse groove 230.

The first and second edges are spaced laterally from each other. Likewise, the third and fourth edges 237, 238 are spaced laterally from each other. The first, second, third and fourth edges are substantially parallel.

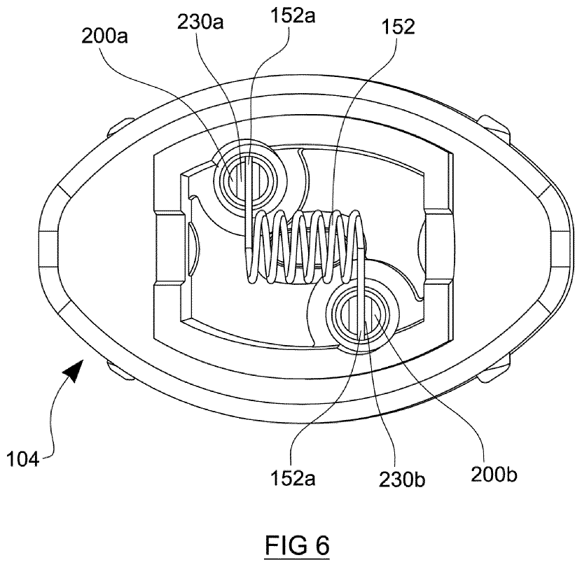

The first and second side walls 233, 234 are moveable between a receiving position (shown in FIGS. 4 and 5) and a fixing position (shown in FIG. 6). In the fixing position, the first and second chamfered portions 235, 236 are spaced closer together than in the receiving position. In other words, the first side wall 233 pivots/hinges about the third edge 237 and the second side wall 234 pivots/hinges about the fourth edge 238. This means that the heating wire 152 may be more easily placed in the transverse groove 230 when the transverse groove 230 is in the receiving position and then held securely in the transverse groove 230 when in the fixing position.

In the receiving position, the transverse groove 230 are tapered such that the first and second side walls 233, 234 are spaced further apart at the first longitudinal end face 211 than at the base 231 of the transverse groove 230. In the receiving position, the side walls 233, 234 of the transverse groove 230 slope inwards towards the longitudinal axis of the main body 210 at a constant angle.

In the receiving position, the first and second chamfered portions 235, 236 of the transverse groove 230 are spaced laterally by 0.3 mm at their closest with a tolerance of ±0.05 mm and the third and fourth edges 237, 238 are spaced laterally by 0.2 mm with a tolerance of ±0.05 mm.

The main body 210 of the electrical contact pin 200 comprises a partition groove 212 on a transverse face 213 of the main body 210. The partition groove extends continuously around the outside of the main body 210. The cross-sectional shape of the main body 210 is a circle, so the partition groove 212 extends around the circumference of a circular cross-section. The partition groove 212 is spaced from the first longitudinal end face 211 in the longitudinal direction by 1 mm.

The depth of the partition groove 212 extends from the transverse side 213 into the main body 210 in a direction perpendicular to the longitudinal direction. The depth of the partition groove is about 0.24 mm.

The partition groove comprises opposed upper wall 214 and lower wall 215 which are spaced apart in the longitudinal direction (shown best in FIG. 5). The upper and lower walls 214, 215 are spaced by 0.2 mm and are substantially parallel to the first longitudinal end face 211.

The upper wall 214 is more proximate the first longitudinal end face 211 than the lower wall 215 and the base 231 of the transverse groove 230 is aligned with the lower wall 215 of the partition groove 212 (i.e. the base 231 of the transverse groove 230 is substantially coplanar with the lower wall 215 of the partition groove 212).

Best seen in FIG. 4, the main body has a uniform portion 216 having a constant transverse cross-sectional area and an upper frustum portion 217 having transverse cross-sectional area which increases in the longitudinal direction (away from the first longitudinal end face 211). The uniform portion 216 is a cylinder and the frustum portion 217 is a truncated cone. The uniform portion 216 and the frustum portion 217 are axially aligned i.e. a longitudinal axis of the uniform portion 216 is substantially collinear with a longitudinal axis of the frustum portion 217. The first frustum (longitudinal) end face is the first longitudinal end face 211. The second frustum (longitudinal) end is longitudinally opposite the first frustum end face of the frustum portion 217 and has a transverse cross-sectional area which is the same area and shape as the transverse cross-sectional area of the uniform portion 216.

The length of the uniform portion 216 in the longitudinal direction is about 3.64 mm. The length of the frustum portion 217 in the longitudinal direction is about 2.37 mm. The frustum portion 217 has a slant angle between its longitudinal axis and a side surface 213 which is about 11 degrees. The frustum portion 217 comprises the partition groove 212.

The uniform portion 216 has a first diameter which is 2.05 mm with a tolerance of +0.05 mm. The first longitudinal end face 211 has a second diameter which is about 1.1 mm.

As shown in FIG. 4, the main body 210 of the electrical contact pin 200 may comprise second longitudinal end face 218 at an opposite end of the main body 210 to the first longitudinal end face 211. The main body 210 comprises a radially protruding flange 219 proximate the second longitudinal end face 218.

The flange 219 has a flange diameter which is 2.5 mm with a tolerance of ±0.02 mm and has a length in the longitudinal direction from the second longitudinal end face 218 of 0.5 mm.

The flange 219 comprises the rotational location feature 220 which takes the form of a flattened surface 221 on a side of the flange 219. This means that the cross-sectional shape of the flange 219 approximately resembles a D-shape. The flattened surface 221 of the protruding portion 219 is tangent to the uniform portion 216 of the main body 210. The flattened surface 221 extends in a direction parallel to the elongate groove axis of the transverse groove 230 and in the longitudinal direction.

FIG. 6 shows the heating wire 152, a first vaporiser electrical contact pin 200a and a second vaporiser electrical contact pin 200b assembled in the component 104. The electrical contact pins 200a, 200b are identical to the electrical contact pin 200 described above.

The electrical contact pins 200a, 200b are in the fixing position described above such that the heating wire 152 is fixed in place. The heating wire 152 has a first end 152a which is fixed in the transverse groove 230a of the first electrical contact pin 200a. The heating wire 152 also has a second end 152b which is fixed in the transverse groove 230b of the second electrical contact pin 200b.

The arrangement shown in FIG. 6 is assembled in the following manner: rotationally locating the electrical contact pins 200a, 200b relative to the aerosol delivery component 104 according to the rotational location feature (not shown in FIG. 6, 220 in FIGS. 4 and 5); press fitting the electrical contact pins 200a, 200b into respective apertures (not shown) of the aerosol delivery component 104; pushing a first end 152a of the vaporiser heating wire 152 into the transverse groove 230a of the first electrical contact pin 200a; pushing a second end 152b of the vaporiser heating wire 152 into the transverse groove 230b of the second electrical contact pin 200b; reducing a respective width of each transverse groove 230a, 230b in order to fix the vaporiser heating wire 152 in the electrical contact pins 200a, 200b.

The step of reducing the width of the transverse grooves 230a, 230b is performed by crimping the contact pins 200a, 200b.

While exemplary embodiments have been described above, many equivalent modifications and variations will be apparent to those skilled in the art when given this disclosure. Accordingly, the exemplary embodiments set forth above are considered to be illustrative and not limiting.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. A vaporiser electrical contact pin for an aerosol delivery component comprising:
a main body comprising a first longitudinal end face; and
a transverse groove in the first longitudinal end face for fixing a heating wire of a vaporiser,
wherein the transverse groove comprises laterally spaced opposing first and second side walls extending from the first longitudinal end face to a base of the transverse groove and wherein the first and second side walls are moveable between a receiving position and a fixing position.

2. (canceled)

3. A vaporiser electrical contact pin according to claim 1 wherein the transverse groove comprises:
a first edge which is formed by the first longitudinal end face of the main body meeting the first side wall; and
a second edge which is formed by the first longitudinal end face of the main body meeting the second side wall,
wherein in the fixing position the first and second edges are spaced closer together than in the receiving position.

4. A vaporiser electrical contact pin according to claim 1 wherein, in the receiving position, the transverse groove is tapered such that the first and second side walls are spaced further apart at the first longitudinal end face than at the base of the transverse groove.

5. A vaporiser electrical contact pin according to claim 3 wherein the first edge comprises a first chamfered portion and the second edge comprises a second chamfered portion.

6. A vaporiser electrical contact pin according to claim 1 wherein the main body of the electrical contact pin comprises a partition groove on one or more transverse faces of the main body.

7. A vaporiser electrical contact pin according to claim 6 wherein the partition groove extends continuously around the outside of the main body.

8. A vaporiser electrical contact pin according to claim 6 wherein the partition grove comprises opposed upper and lower walls which are spaced apart in the longitudinal direction and are substantially parallel to the first longitudinal end face and wherein the base of the transverse groove is aligned with the partition groove in a longitudinal direction.

9. A vaporiser electrical contact pin according to claim 8 wherein the base of the transverse groove is aligned with the upper or lower wall of the partition groove in the longitudinal direction.

10. A vaporiser electrical contact pin according to claim 1 wherein the electrical contact pin comprises a rotational location feature.

11. An aerosol delivery component comprising:
at least one vaporiser electrical contact pin according to claim 1; and
a vaporiser heating wire,
wherein the vaporiser heating wire is fixed within the transverse groove of the at least one vaporiser electrical contact pin.

12. An aerosol delivery component according to claim 11 which is a consumable component for receipt in a smoking substitute device.

13. A method of assembling an aerosol delivery component comprising a vaporiser electrical contact pin according to claim 1 and a vaporiser heating wire, the method comprising:
pushing a first end of the vaporiser heating wire into the transverse groove of the vaporiser electrical contact pin;
reducing a width of the transverse groove in order to fix the vaporiser heating wire in the vaporiser electrical contact pin.

14. A method according to claim 13, the method further comprising press fitting the vaporiser electrical contact pin into an aperture of the aerosol delivery component.

15. An aerosol-delivery system comprising an aerosol delivery component according to claim 11 and a device comprising a power source.
